(19) United States
(12) Patent Application Publication
(10) Pub. No.: US 2016/0030409 A1
(43) Pub. Date: Feb. 4, 2016

(54) METHOD FOR RELIEVING CHRONIC PAIN

(71) Applicant: Grigory Karmy, Toronto (CA)

(72) Inventor: Grigory Karmy, Toronto (CA)

(21) Appl. No.: 14/814,115

(22) Filed: Jul. 30, 2015

Related U.S. Application Data
(60) Provisional application No. 62/030,795, filed on Jul. 30, 2014.

(51) Int. Cl. A61K 31/445; A61K 9/00

(52) U.S. Cl. CPC A61K 31/445; A61K 9/0021

(57) ABSTRACT

The method is for use in a part of a mammal having one or more tender points and comprises a dministering a fluid, by a needleless, pressure-based injector, to substantially all of the tender points in the part. The volume of the fluid that is administered to each of the tender points can be about 0.5 cc. The operating pressure of the injector can be about 150 psig. The fluid can be a local anesthetic such as ropivacaine.

### CROSS-REFERENCE TO RELATED APPLICATIONS

This application claims priority to U.S. Provisional Patent Application Ser. No. 62/030,795 filed Jul. 30, 2014, which is herein incorporated by reference.

### BACKGROUND OF THE INVENTION

1. Field of the Invention

The present invention relates to the field of pain treatment.

2. Prior Art.

Many people suffer from chronic non-malignant pain due to various diseases, such as fibromyalgia, osteoarthritis, carpal tunnel syndrome, migraines, irritable bowel syndrome, spastic bladder syndrome, stroke, multiple sclerosis and Parkinsons. Local anesthetics, such as ropivacaine, are sometimes injected around nerves, into joints and into trigger points to provide pain relief. Such procedures are administered by needle injection on or near the area of pain, but are not always effective in relieving pain.

### SUMMARY OF THE INVENTION

Forming one aspect of the invention is a method of relieving chronic non-malignant pain in a part of a mammal having one or more tender points, the method comprising: administering a fluid, by a needleless, pressure-based injector, to substantially all of the tender points in the part.

Further aspects of the invention will become apparent from the following description.

### DETAILED DESCRIPTION OF THE PREFERRED EMBODIMENTS

In a trial experiment, a middle-aged female patient, approximately in her late 40s to early 50s, was suffering from chronic non-malignant pain due to fibromyalgia and osteoarthritis. The patient was treated with the anesthetic Naropin® (generically known as ropivacaine), by injecting 0.5 cc of the anesthetic, by needle, into most tender points above and below the knees. Naropin was administered at a concentration of 5 mg/ml. No pain relief was realized by the patient as a result of the treatment.

Subsequently, after sufficient time had elapsed for the previously injected Naropin to leave the patient's body, 0.5 cc of Naropin was once again administered, at a concentration of 5 mg/ml, using the Medical International Technology, Inc. (MIT) Med-Jet® H™ needleless, pressure-based injector. The injector was set at a pressure of 150 psig. For knee pain, most tender points, approximately five to eight, above and below the knees were injected. For hip pain, most tender points, approximately five, above the trochanteric bursa were injected. For back pain, most tender points, approximately ten on each side of the back, around the sacroiliac joints were injected. A single injection was administered for each tender point selected for injection. The administration of the local anesthetic by needleless injection left painful welts at each injection site, which took approximately two to three weeks to disappear. When the welts had disappeared, the tender points were no longer tender and pain was diminished.

Further testing was carried out at chronic pain clinics. The patients selected for treatment did not respond well to more traditional injections and were therefore interested in trying a new approach. 25 patients were followed. Responders were treated two to ten times while non-responders were usually treated once only.

In terms of the devices used to treat, Medjet HIII from MIT Canada was used on most patients but in some cases, Mesojet from MIT Canada and Comfort-In Pain Free Injection System from Eternity Healthcare were used. Unless otherwise specified local anesthetic was used with each injector.

Results:

Out of 25 patients treated with needle free injectors 13 had a response superior to trigger point injections and nerve blocks in all areas treated while 8 had superior response to trigger point injections in some but not all areas treated. 3 patients did not respond to treatment.

Case 1:

SL was a female with osteoarthritis and fibromyalgia who complained of back pain, neck and shoulder pain. She had two treatments with radiofrequency ablation for her back. The first one worked very well for nine months, but second one only exacerbated the pain. Landmark based nerve blocks and trigger point injections were helpful for neck and shoulder pain. They also helped back pain initially, but unfortunately became less effective over time with benefit lasting 3-4 days. The patient's back was subsequently treated with Medjet which lead to improvement which lasted for 2 weeks. The patient continued to be seen every 1-3 weeks for the subsequent 6 months and she continued to respond to the treatment. The response was there irrespective of whether she was treated with Medjet or Mesojet. During some of the visits nerve blocks were also administered and also in some areas treatment with Medjet with needle attachment was superior to treatment with regular nozzle.

Case 2

LL is a female with widespread pain due to fibromyalgia and osteoarthritis. Her pain was managed by nerve blocks and trigger point injections. She would normally get 4-7 days of relief after each treatment. She was subsequently treated with Medjet for her hip pain. The pain relief lasted for about 6 weeks. Other parts of her body such as arms, back, shoulders and back were treated with either Medjet or Mesojet during subsequent visits. The responses ranged from 2-6 weeks. Also Comfort In Injection System was used on the right shoulder with similar results. She was seen and treated every two to three weeks for one year and while some areas stopped responding to treatment after about 6 month, others continued to respond.

Case 3

AT is a female with wide spread pain due to fibromyalgia. Her response to nerve blocks and trigger point injections was very erratic. Most of the time the relief would last for two days, however on occasion the response would last as long as two weeks. When she started treatments with Medjet, the responses became more consistent. The relief for lower back pain would last for 1-2 weeks while relief for neck and shoulder pain would last for 4 days to one week. She continued to be seen every 2-6 weeks for the subsequent 6 months and treated with either Medjet or Mesojet. On occasion she also received nerve blocks on the same visit. The response continued to be superior to treatment with trigger point injections alone. Normal saline instead of local anesthetic was also injected on trial basis. The duration of response was similar to response when local anesthetic was used.

In summary, a number of patients responded far longer to treatment with needle free injector than to treatment using needles. Without intending to be bound by theory, it is postulated that localized area of high pressure generated by the injector is the cause for this additional benefit:
the benefit from needleless injection is much longer than the benefit from trigger point needle injections,, even in cases in which the amount of medication administered and the distribution of treatment sites is comparable; and
administration of normal saline in some of the patients also lead to significant pain relief.

Whereas 150 psig was used in the initial experiment mentioned above, different pressure settings are plausible, depending on skin thickness, severity of pain, tenderness and/or sensitivity to pain of the area being injected, and other relevant factors. Furthermore, whereas 0.5 cc of Naropin was administered in the trial experiment at a concentration of 5 mg/ml, it will be understood that different amounts and concentrations of the drug may be used, depending on the severity of the pain, the size of the patient, the age of the patient, and other relevant factors. It will further be appreciated that local anesthetics, other than ropivacaine, can be used. Further, whereas welts formed in the skin of the initial test patient, the advantage was attained without the formation of welts on many occasions.

It is to be understood that what has been described is an exemplary embodiment of the invention. The scope of the claims should not be limited by the embodiment set forth above, but should be given the broadest interpretation consistent with the description as a whole.

What is claimed is:

1. A method of relieving chronic non-malignant pain in a part of a mammal having one or more tender points, the method comprising:
administering a fluid, by a needleless, pressure-based injector, to substantially all of the tender points in the part.

2. The method of claim 1, wherein the fluid is an inert fluid safe for mammalian injection.

3. The method of claim 2, wherein a volume of the fluid that is administered to each of the tender points is about 0.5 cc.

4. The method of claim 1, wherein an operating pressure of the injector is about 150 psig relative to atmospheric pressure.

5. The method of claim 1, wherein the operating pressure of the injector is such that welts form in the skin at the injection sites.

6. The method of claim 1, wherein the fluid is a local anesthetic.

7. The method of claim 6, wherein the local anesthetic is ropivacaine.

8. The method of claim 7, wherein a volume of the fluid that is administered to each of the tender points is about 0.5 cc and the concentration of the ropivacaine that is administered to each of the tender points is about 5 mg/ml.
